(19) United States
(12) Patent Application Publication
(10) Pub. No.: US 2012/0149584 A1
(43) Pub. Date: Jun. 14, 2012

(54) METHODS OF DIAGNOSING AND TREATING MICROBIOME-ASSOCIATED DISEASE USING INTERACTION NETWORK PARAMETERS

(76) Inventors: Bernat Olle, Cambridge, MA (US); Jonathan Robert Behr, Boston, MA (US); W. Edward Martucci, Norwood, MA (US); Daphne Zohar, Boston, MA (US)

(21) Appl. No.: 13/391,352

(22) PCT Filed: Aug. 20, 2010
(86) PCT No.: PCT/US10/46184

§ 371 Date: Feb. 20, 2012

Related U.S. Application Data
(60) Provisional application No. 61/235,889, filed on Aug. 21, 2009.

(51) Int. Cl. C40B 20/00; G06G 7/58; C12Q 1/68

(52) U.S. Cl. 506/2; 435/6.12; 703/11

(57) ABSTRACT

Methods of diagnosing and treating microbiome-associated disease or improving health using interaction network parameters are provided. Methods are provided to analyze interaction networks between microbes, and between microbes and the host, to determine important (e.g. “highly-connected”) organisms or molecules as determined by various network parameters. Methods are provided including and beyond correlation to use these “highly-connected” organisms or molecules as targets for modulation or as therapeutic agents to improve health.

### FIELD OF THE INVENTION

The present invention is generally in the field of microbiome-associated diseases and relates in particular to methods of diagnosing and treating a microbiome-associated disease or improving health using interaction network parameters

### BACKGROUND OF THE INVENTION

Animals, including humans, host a multitude of microbes (collectively referred to as the host's microbiota) in anatomical locations including the mouth, esophagus, stomach, small intestine, large intestine, caecum, colon, rectum, vagina, skin, nasal cavities, ear, and lungs. These locations offer environments with varying conditions of pH, redox potential, presence of host secretions, and contact with the immune system, among other factors, where intense competition among bacteria leads to specialization in certain functional roles. Furthermore, the host exerts selective pressure for functional redundancy to prevent loss of key functions. As a result, groups of bacterial commensals that share a specialization or function are established. These can be generally referred to as functional niches. Elucidation of the functional roles of such niches has been the focus of recent research which has established that, collectively, the human microbiota is responsible for a multitude of critical processes, including metabolism of carbohydrates and proteins, maturation of the immune system, formation and regeneration of the epithelium, fat storage, production of hormones, metabolism of xenobiotics, production of vitamins, and protection from pathogen infections, among others (Hooper L V, Gordon J L Science. 2001; 292:1115; Rakoff-Nahoum S, Paglino J, Eslami-Varzaneh F, Edberg S, Medzhitov R. Cell. 2004; 118:229; Backhed F, et al. Proc. Natl. Acad. Sci. U.S.A. 2004; 101:15718; Stappenbeck T S, Hooper L V, Gordon J I. Proc. Natl. Acad. Sci. U.S.A. 2002; 99:15451; 1 Sonnenburg J L, Angenent L T, Gordon J I. Nat. Immunol. 2004; 5:569; Hooper L V, et al. Science. 2001; 291:881)

Among these groups of commensals, complex genetic, transcriptomic proteomic, and metabolic networks are established, wherein certain key bacteria stand out because of being highly connected with other members of the network or because of occupying central locations in such networks.

Despite the plausible importance of specific, highly-connected, selected members of the microbiota in health and disease, there is a lack of approaches to identify them and characterize their interactions with other members of the microbiota. Existing genomic and metagenomic methods are limited by the difficulties in studying the functional ecology of the symbionts.

The most complex interactions between the microbiota to date are modeled solely based on degree of evolutionary similarity (such analyses usually presented by phylogenetic trees), and thus do not report on the importance (i.e. centrality, connectedness, etc) of each piece of the network, or an entire network as a whole.

It is therefore an object of this invention to provide approaches that allow construction and analysis of important associations and network parameters of complex bacterial networks, which in turn enable identification of either key organisms of the human microbiota involved in health and disease, key microbiota-derived mediators of health and disease, or key microbiota modulators involved in health and disease.

It is a further object of the invention to utilize such network parameters to diagnose a microbial-related condition or provide a therapeutic strategy for a microbial-related condition.

### SUMMARY OF THE INVENTION

Methods of diagnosing and treating microbiome-associated diseases or improving health using interaction network parameters are provided. Methods are provided to analyze interaction networks between microbes, and between microbes and the host, to determine important (e.g., “highly-connected”) organisms or molecules as determined by various network parameters. Methods are provided including and beyond correlation to use these important (e.g., “highly-connected”) organisms or molecules as targets for modulation or as therapeutic agents to improve health. Products are also provided containing microbiota modulators, probiotics, or other therapeutic agents derived from these important “highly-connected” organisms or molecules for the improvement of health.

In one embodiment, a method for developing microbiota modulators for the improvement of health is provided, comprising (i) analyzing a biological interaction network within a superorganism which includes at least one microbial derived component (ii) selecting a node or edge in the network based on one or more network parameters, and (iii) developing modulators of the node or edge

In another embodiment, a method for developing diagnostics for the determination of a physiological state is provided, comprising (i) analyzing a biological interaction network within a superorganism which includes at least one microbial derived component, (ii) selecting one or more node and/or edge in the network based on one or more network parameters, and (iii) developing a diagnostic to measure the edge or node

In one embodiment, identification of network parameters comprising topographical (pattern) parameters enables identification of key members of the microbiota associated with a health or a disease state. The network parameters may be selected from parameters including, but not limited to, physical proximity, relative prevalence, connectivity, evolutionary similarity, density, geodesics, centralities, Small World, structural equivalence, Cluster coefficient, Krackhardt E/I Ratio, Krebs Reach & Weighted Average Path Length, distances, flows, shared neighbors, and shortest path length.

In one embodiment, identification of network parameters comprising a process (functional parameters), such as covariance, enables identification of a key member of the microbiota associated with a health or a disease state.

The properties of high connectivity with other members and centrality in the networks may be a surrogate for a bacteria's key role in health as well as in disease conditions. Methods that enable analysis of interaction networks between microbes, and between microbes and the host, to determine “highly-connected” organisms or molecules, therefore may be used to diagnose and treat a microbiome-associated disease or to improve health.

### DETAILED DESCRIPTION OF THE INVENTION

### I. Definitions:

The term “microbiota” refers, collectively, to the entirety of microbes found in association with a higher organism, such as a human. Organisms belonging to a human's microbiota may generally be categorized as bacteria, archaea, yeasts, and single-celled eukaryotes, as wells as viruses and various parasites such as Helminths.

The term “microbiome” refers, collectively, to the entirety of microbes, their genetic elements (genomes), and environmental interactions, found in association with a higher organism, such as a human.

The term “commensal” refers to organisms that are normally harmless to a host, and can also establish mutualistic relations with the host. The human body contains about 100 trillion commensal organisms, which have been suggested to outnumber human cells by a factor to 10.

The term “microbial derived component” refers to a component consisting of, emanating from, or produced by members of the microbiota. The component can be, for example, a microbe, a microbial protein, a microbial secretion, or a microbial fraction.

The term “anatomical niche” describes a region of a host, such as the gut, the oral cavity, the vagina, the skin, the nasal cavities, the ear, or the lungs. The term may also refer to a structure or sub-region within any of these regions, such as a hair follicle or a sebaceous gland in the skin.

The term “functional niche” describes a group of organisms, such as microbes, that specialize in a certain function, such as carbohydrate metabolism or xenobiotic metabolism.

The term “network” refers to a constructed representation of components (host or microbial-derived components) describing the connection of the components by various methods.

The term “node” refers to a terminal point or an intersection point of a graphical representation of a network. It is the abstraction of an element such as an organism, a protein, a gene, a transcript, or a metabolite.

The term “edge” refers to a link between two nodes. A link is the abstraction of a connection between nodes, such as covariance between the nodes.

The term “motif” refers to a pattern that recurs within a network more often than expected at random.

The term “highly connected organism” refers to a key functional member of the microbiota that has edge connections to a large number of nodes in the network. For example, a bacterial species may perform biotransformations of numerous metabolites, thus plausibly influencing host metabolism and host health.

The term “modulating” as used in the phrase “modulating a microbial niche” is to be construed in its broadest interpretation to mean a change in the representation of microbes in a bacterial niche of a subject. The change may be an increase or a decrease in the presence of a particular species, genus, family, order, class, or phylum. The change may also be an increase or a decrease in the activity of an organism or a component of an organism, such as a bacterial enzyme, a bacterial antigen, a bacterial signaling molecule, or a bacterial metabolite.

The term “metagenomics” refers to genomic techniques for the study of communities of microbial organisms directly in their natural environments, without requiring isolation and lab cultivation of individual species.

### II. Microbiota Modulators

Interventions known to modulate the microbiota include antibiotics, prebiotics, probiotics, and synbiotics. Antibiotics generally eradicate the microbiota without selectivity as a byproduct of targeting an infectious pathogen. In contrast, nutritional approaches involving live organisms (probiotics), non-digestible food ingredients that stimulate the growth or activity of bacteria (prebiotics), or combinations of both (synbiotics), are more benign but exert a moderate beneficial effect on the host. Other therapeutic modalities that can be used as microbiota modulators include non-antibiotic small molecule modulators, biologics, DNA or RNA-based agents, and polymers. These approaches can directly target microbes (such as those mentioned above), or can modulate microbes indirectly through perturbation of host physiology (such as pharmaceutical agents and nutritional components known to affect host physiology and biochemical pathways).

### III. Types of Network

The networks may include bacterial interaction networks, where all the nodes in the network correspond to bacterial organisms or bacterial molecules; bacterial-host interaction networks, where the nodes in the network correspond to both bacterial cells or molecules as well as host cells or molecules; whole-organism level interaction networks, where the nodes in the network correspond to interrelated molecules within one organism; biochemical interaction networks, such as metabolic, regulatory or signal transduction pathways, where the nodes are molecular species in a cell or in a larger system; or some combination of the above.

Networks can be constructed by one skilled in the art using known methods. An example of the construction of a metabolic network is described in Borenstein E and Feldman M W, 2009, J. Comput. Biol. 16(2): 191-200. An example of the construction of topological species networks is described in Naqvi A et al, 2010, Chem. Biodivers. 7(5): 1040-50.

### IV. Node Components

The elements that constitute the nodes may be an organism or group of organisms selected from a niche, a strain, a species, a genus, a family, an order, a class, or a phylum. The elements that constitute the nodes may also be selected from a protein, a gene, an RNA transcript, a carbohydrate, a lipid, a metabolite, a small molecule, a vitamin, a gas, an ion, or a salt. The elements that constitute the nodes may also be selected from functions of the microbiome, such as effects on host genes, cellular readouts, cell fates or differentiation, or perturbations of metabolic pathways or effector molecules.

### V. Edge Components

The elements that constitute the edges may be selected from biological interactions such as transformations, catalysis, complex formation, signal transfer, regulation by protein-protein interaction, protein phosphorylation, regulation of enzymatic activity, production of secondary messengers, or any other biological process. The elements that constitute the edges may be selected from molecules including a protein, a gene, an RNA transcript, a carbohydrate, a lipid, a metabolite, a small molecule, a vitamin, a gas, an ion, or a salt. The edges of the network may be further described by parameters calculated from properties of the elements. These properties of the elements can be selected from properties such as, weight, connectivity, or other measures reflecting values specific to each edge.

Elements of selecting and modifying edges in a network are known to one skilled in the art. An example of selecting edges can be found in Naqvi A et al, 2010, Chem. Biodivers. 7(5): 1040-50. The edge can be a correlation between bacterial species or taxa in a microbiome sample, including co-occurrence. The intensity or weight of the edge can be determined by counting either the number of individual samples where both species are present above a certain abundance threshold, or by using the abundance information of various interacting nodes.

### VII. Network Parameters and Motifs

In one embodiment, identification or calculation of network parameters comprising topographical (pattern) parameters enables identification of a key member of the microbiota associated with a health or a disease state. The network parameters may be selected from measures such as physical proximity, relative prevalence, connectivity (i.e. number of connections, strength of connections, distance of connections, etc.), evolutionary similarity, density, geodesics, centralities, Small World, structural equivalence, Clustering coefficient, Krackhardt E/I Ratio, Krebs Reach & Weighted Average Path Length, distances, flows, shared neighbors, and shortest path length. For example, in one embodiment, the network parameter is a connection between two components (i.e. a path between the two components). In another embodiment, the parameter measures the degrees of a node (i.e. the number of edges incident on the node). In another embodiment, the parameter measures the shortest path length (whether a node is reachable through a path starting from a second node, and if so, the minimum number of edges traveled). In another embodiment, the parameter is a measure of eccentricities (the length of the path from a given node to any other reachable node that has the largest length among all shortest paths). In another embodiment, the parameter is a measure of betweenness (the number of node pairs (n1, n2) where the shortest path passes through a selected node). In yet another embodiment, the parameter is a clustering coefficient (a measure that assesses the degree to which nodes tend to cluster together). In another embodiment, the derivatives of the above network parameters are used including means or medians of the parameters (e.g., the node is selected with the average shortest path length to any other node).

In another embodiment, identification or calculation of network parameters comprising a process (functional parameters), such as covariance, which enables identification of a key member of the microbiota associated with a health or a disease state.

In another embodiment, identification or calculation of a network parameter that quantifies the degree of conservation across two or more bacterial species of a metabolic pathway, a signal transduction pathway, a protein complex or protein interaction, or a protein-metabolite interaction enables identification of a key member of the microbiota associated with a health or disease state. In a preferred embodiment, identification of the degree of conservation parameter involves the steps of (i) aggregating in one database data comprising a set of protein-protein interactions (measured by methods such as affinity purification or yeast two hybrid, as outlined below) and protein-metabolite interactions (such as enzymatic biotransformations, allosteric interactions, etc, which may be measured by methods known in the art such as enzymatic assays, and fluorescence assays) of two or more bacterial organisms, (ii) quantifying the number of interactions shared by the two or more organisms, and (iii) selecting the interactions shared by two or more organisms. The shared interactions conserved across species may indicate that the proteins or metabolites perform a key role for the organism's survival. Alternatively, a low degree of conservation parameter (for example, corresponding to a bacterial organism that does not share a protein-protein interaction, or a protein-metabolite interaction with any other or with most other bacterial organism of the microbiota), may indicate that the interaction can be specifically interrupted with an intervention (e.g. a drug or a dietary component) with little or no effect to the host or to the rest of the microbiota. The interruption may be desirable to limit the growth of a bacterial species overrepresented in a disease state (for example, limiting the growth of Firmicutes in an obese patient).

In one embodiment, identification of a network motif enables identification of a key member of the microbiota associated with a health or a disease state. The motif may be selected from a chain motif (a sequence of nodes each connecting to the next one in the sequence), a cycle motif (a chain of nodes, with the last node in the chain connecting to the first node), a complete two layer motif (two sets of distinct nodes, with every node in the first set connecting to every node in the second set), a Negative auto-regulation motif (for example, a transcription factor repressing its own transcription), a Positive auto-regulation motif (for example, a transcription factor enhancing its own rate of production), a Feed-forward loop motif (a chain of distinct nodes, with the first node connecting to the last node; See for example Mangan et al, PNAS, 2003. 100(21): p. 11980-5), a Single-input module motif (wherein a single regulator regulates a set of genes with no additional regulation), and a Dense overlapping regulon motif (wherein several regulators control a set of genes in a combinatorial fashion).

The identification of motifs can be performed by methods known to one skilled in the art. Examples of algorithms efficient for finding motifs in biological networks include FANMOD (Wernicke S and Rasche F, 2006 Bioinformatics 22:1152) and MAVISTO (Schreiber F and Schwobbermeyer H, 2005 Bioinformatics 21:3572). An example of applying motif-fitting algorithms to such a network is described in Naqvi A et al, 2010, Chem. Biodivers. 7(5): 1040-50.

### VIII. Methods of Analyzing a Biological Interaction Network

The source material for analysis of an existing network and/or construction of a network can be collected from studies using humans, animals, or computational means (i.e. existing databases). The source material can be genomic, macromolecular (i.e. carbohydrate, protein, lipid, nucleic acid), small molecule based (e.g. metabolites), or other components as described in detail above. If collected from a living source (i.e. a human or animal), the material can be isolated and purified from natural tissues and biofluids, such as skin, urine, feces, saliva, mucus, tissue biopsies, and others described in detail below. In one embodiment, urine or feces from a subject are collected, and the genomic and metabolic content isolated and analyzed to build a network.

### Methods to Analyze the Genetic Content of a Biological Network

In one embodiment, the method involves screening of 16srRNA genes by PCR, which enables characterization of microorganism at the phylum, class, order, family, genus, and species level. The sequences of the 16srRNA gene contain hypervariable regions which can provide specific signature sequences useful for bacterial identification. (Schloss and Handelsman, Microbiol. Mol. Biol. Rev., 2004, 68: 686-691). Sequence hits can be screened using searching algorithms and databases (e.g. BLAST) to determine taxonomic information.

In another embodiment, a high-throughput “metagenomic” sequencing method is used, such as pyrosequencing. Genetic features are identified by isolating a sample from a bacterial niche, extracting the DNA of the bacterial fraction, cloning the DNA in a vector that replicates in a cultured organism, introducing the vectors in bacteria to create a metagenomic library, and identifying phylogenetic markers in the DNA sequences of the library that link the cloned sequences to the probable origin of the DNA and the probable functions encoded by such genes. The method identifies genes that are either over-represented or under-represented in the bacterial population. Furthermore, the method enables the sequencing of genetic material from uncultured communities of microbial organisms directly in their natural environments, bypassing the need for isolation and lab cultivation of individual species (Handelsman et al. (1998). Chem. & Biol. 5: 245-249).

In another embodiment, “gene chips” containing an array of genes that respond to extracted mRNAs produced by cells (Klenk et al., 1997 Nature, 390, 364-370) can be used. Many genes can be placed on a chip array and patterns of gene expression, or changes therein, can be monitored.

### Methods to Analyze the Proteomic Content of a Biological Network

In one embodiment, proteomic techniques are used to analyze a biological network. Proteomic methods yield a measurement of the production of proteins of an organism (Geisow, 1998 Nat. Biotechnol. 16:206). Proteomic measurements generally involve a step consisting of a protein separation method, such as 2D gel-electrophoresis, followed by a chemical characterization method, generally a form of mass spectrometry.

In one embodiment, an immune response by the host can be used as a reporter to identify a key microbial protein. A microbial cell surface antigen characteristic of a certain niche can be detected by administering a strain to a host and isolating a serum antibody against the strain secreted by the host. For example, a lambda phage expression library of total cecal bacterial DNA can be constructed and then screened using serum IgG from a patient suffering from colitis. Positive clones can be collected and rescreened for verification. At the end of the process, the remaining clones can be sequenced. The sequences can be matched against clones in reference datasets, such as GenBank, and homology with existing bacterial proteins is established. Additionally, a recombinant version of the microbial antigen-binding antibodies identified, or relevant fragments of the antibody, or relevant epitope sequences introduced into a recombinant construct, may be expressed in a recombinant system (e.g. E. coli, yeast, or a Chinese Hamster Ovary cells), purified and used as a microbiota modulator.

In another embodiment, phage display technology is used to purify and characterize key proteins from a bacterial network. In this method, bacterial proteins are displayed on the surface of the bacteriophage virion. Display is achieved by fusion of a bacterial protein or library of proteins of interest to any virion proteins such as the pIII and pVIII proteins. Filamentous phage virion proteins are secreted by translocation from the cytoplasm via the Sec-dependent pathway and anchored in the cytoplasmic membrane prior to assembly into the virion (Jankovic et al., Genome Biol. 2007; 8(12): R266). In this fashion, all types of bacterial secreted proteins, including receptors, adhesions, transporters, complex cell surface structures, secreted enzymes, toxins, and virulence factors, can be identified. In order to deduce whether a protein is likely to be secreted, several methods can be used, including SignalP 3.0, TMHMM 2.0, LipoPred, or PSORT (Bendtsen J D, Nielsen H, von Heijne G, Brunak S: J Mol Biol 2004, 340:783-795). These methods deduce secreted proteins from a completely sequenced genome by using a range of algorithms that identify signal sequences and transmembrane α-helices, which are characteristic of secreted proteins. In another embodiment, a key interaction between a bacterial protein and a host protein, or between two bacterial proteins is identified by methods known in the art such as affinity purification (in which case a complex formed by the two proteins can be identified, See for example Gavin et al, Nature, 440, 631-636, 2006), or yeast two hybrid methods (in which case numerous complexes formed by pairs of proteins can be identified in a high throughput manner).

### Methods to Analyze the Metabolic Content of a Biological Network

In one embodiment, the method used to analyze a biological network uses metabolomic or metabonomic approaches. These methods have been developed to complement the information provided by genomics and proteomics by analyzing metabolite patterns (See, for example, Nicholson et al., 1999 Xenobiotica 29 (11): 1181-9). Metabonomics is based on the application of 1H NMR spectroscopy and mass spectrometry to study the metabolic composition of biofluids, cells, and tissues, in combination with use of pattern recognition systems and other chemoinformatic tools to interpret and classify complex NMR-generated metabolic data sets.

### Methods to Analyze the Glycan Content of a Biological Network

In one embodiment, the method used to analyze a biological network uses “Glycoinic” methods. These methods can be used to comprehensively study glycomes (the entire complement of sugars, whether free or present in more complex molecules, of an organism). The tool used most often in glycomic analysis is high resolution mass spectrometry. In this technique, the glycan part of a glycoprotein is separated from the protein and subjected to analysis by multiple rounds of mass spectrometry. Mass spectrometry can be used in conjunction with HPLC. Other techniques include lectin and antibody arrays, as well as metabolic and covalent labeling of glycans.

### Methods to Analyze the Lipid Content of a Biological Network

In one embodiment, the method used to analyze a biological network uses “lipidomic” approaches. Lipid profiles pertaining to biological networks of the invention can be studied with a number of techniques that rely on mass spectrometry, nuclear magnetic resonance, fluorescence spectroscopy and computational methods. These techniques involve steps of lipid extraction (using solvents well known in the art), lipid separation (typically using Solid-phase extraction (SPE) chromatography, and lipid detection (typically using soft ionization techniques for mass spectrometry such as electrospray ionization (ESI) and matrix-assisted laser desorption/ionization (MALDI)

### Types of Samples Analyzed

Biofluids such as urine, blood, plasma, saliva, sputum, mucus, and CSF, as well as fecal samples, hair samples, skin samples, and tissue biopsies or homogenates may be used for testing.

Computer-Based Methods to Identify Relationships within a Biological Network

Computational methods for modeling and analysis of biological networks are known in the art. Complex data generated by any of the methods above including gene, RNA, protein, metabolite, glycan, and lipid information can be analyzed by computational “pattern recognition.” Pattern recognition classifies data patterns based either on a priori knowledge or on statistical information extracted from the patterns. Pattern recognition methods involve schemes for classifying or describing observations, relying on the extracted features. The classification or description scheme can be based on the availability of a set of patterns that have already been classified or described. This set of patterns is termed the training set, and the resulting learning strategy is characterized as supervised learning. Learning can also be unsupervised, when the system is not given a priori labeling of patterns, instead it itself establishes such classes based on statistical patterns. Examples of unsupervised pattern recognition methods include principal component analysis (PCA) (Kowalski et al, 1986), hierarchical cluster analysis (HCA), and non-linear mapping (NLM) (Brown et al., 1996; Farrant et al., 1992).

Data may also be analyzed by building probabilistic Bayesian models, linear algebraic equation models, partial least squares models, or Boolean models.

Data may also be analyzed by sequence similarity methods that identify orthologous proteins from two different organisms, by graph comparison algorithms that identify gene duplications (See for example Sharan and Ideker, Nat. Biotechnol. 24, 427-433, 2006), and by several other tools available online for comparing sets of interactions (See for example Kelley, PNAS, 100, 11394-11399, 2003, or Sharan et al, PNAS, 102, 1974-1979, 2005).

An example of computing network properties in biological networks is described in Naqvi A et al, 2010, Chem. Biodivers. 7(5): 1040-50. Network properties that are calculated include the degree distribution (i.e. the number of neighboring connections of a node), the average network diameter (i.e. the average shortest path between all pairs in the network), and the average clustering coefficient. (i.e. the probability of two nodes each connected individually to a third node are themselves connected). Additionally, network operations can be performed that involve overlapping one or more independent networks, sub-networks, motifs or patterns within a network in order to find the intersection, union, and difference of particular nodes and edges. This information can be used to determine a “core” set of parameters for the model that would apply across individuals.

Experimentally-determined network models can be fit to existing models. Naqvi A et al, 2010, Chem. Biodivers. 7(5): 1040-50 describes the practice of fitting a biologically-generated network model with known random graph models, such as the Erdos-Renyi. Measures of structural similarities between two networks can be applied, such as RGF-distance and GDD-agreement, both of which are described in the art.

### Methods of Selecting a Node or Edge Based on a Network Parameter

Important nodes or edges may be selected based on the calculated network parameters. In one embodiment, the important node or edge is identified by the calculated parameter being the highest value in the network. In another embodiment, the important node or edge is identified by the calculated parameter being in the top X percent of the rank ordered parameter values, where X can be 1%, 2%, 5%, or 10%. In one embodiment, the important node or edge is identified by the calculated parameter being the lowest value in the network. In another embodiment, the important node or edge is identified by the calculated parameter being in the bottom X percent of the rank ordered parameter values, where X can be 1%, 2%, 5%, or 10%. In another embodiment, the node or edge is selected due to the fact that the network parameter for that node or edge is an outlier compared to the parameter for the other nodes or edges respectively (e.g., lying between two modes in a bimodal distribution.

Examples of selecting nodes or edges in networks are known in the art. One example of selecting nodes and edges in a biological topological network is described in Naqvi A et al, 2010, Chem. Biodivers. 7(5): 1040-50.

### In Vivo Confirmation of Identified Relationships for Validation of Critical Nodes

Perturbation of one or more nodes or edges in the network can help to build or refine a network, or validate a network. Perturbation of a network can include altering the presence, level, function, magnitude, or intensity of a node or edge. In one embodiment, one or more nodes or edges are altered in order to refine the construction or understanding of a network. In one embodiment, the node or edge can be perturbed by a microbiome modulator, such as a prebiotic, probiotic, antibiotic, bacteriocin, or other drug or nutrient. The corresponding response of one or more of the nodes in the model is used to refine the network. In one embodiment, the covariance of nodes or edges in response to a perturbation is used to define the connectivity of those nodes or edges.

In one embodiment, the importance of a node or edge can be validated by removing or decreasing the amount or function of a node or edge. In one embodiment, in order to validate the importance of a relationship identified by the methods outlined above, genetic deletions of selected nodes (if those nodes are genes) can be made, and the resulting changes in gene expression, protein expression, and metabolite profiles, as well as phenotype, can be observed. Several approaches known in the art can be used to validate the biological relevance of an identified relationship. Genetic techniques such as knockouts by homologous recombination may be used. Use of RNAi techniques may also enable the rapid assessment of gene function and regulation, as well as other knockout techniques (See Ding et al, Cell, 122, 473-483, 2005). In another embodiment, small molecule inhibitors or protein inhibitors such as antibodies or soluble receptors may be used to remove or decrease the function of a node or edge.

In another embodiment, the importance of a node or edge can be validated by supplementing the amount or function of a node or edge. In one embodiment, a gene or genetic construct (such as a plasmid) is inserted into the network (e.g., by viral transfection, gene gun, naked addition, or other methods known in the art). In another embodiment, a protein, lipid, carbohydrate, small molecule, gas, ion, or salt, is added to the system. In another embodiment, a live organism is added to the system.

IX Diseases and Conditions Associated with Altered Microbial Networks

Disease states may exhibit either the presence of a novel microbe(s), absence of a normal microbe(s), or an alteration in the proportion of microbes. Disease states may also have substantially similar microbial populations as normal states, but with a different microbial function or a different host response to the microbes due to environmental or host genetic factors. Additionally, similar microbial functions may be identified, but the network topology or dynamic response may be altered in a disease state or condition versus a healthy state.

Recent research has established that disruption of the normal equilibrium between a host and its microbiota, generally manifested as a microbial imbalance, is associated with, and may lead to, a number of conditions and diseases. These include Crohn's disease, ulcerative colitis, obesity, asthma, allergies, metabolic syndrome, diabetes, psoriasis, eczema, rosacea, atopic dermatitis, gastrointestinal reflux disease, cancers of the gastrointestinal tract, bacterial vaginosis, neurodevelopmental conditions such as autism spectrum disorders, and numerous infections, among others. For example, in Crohn's disease, concentrations of Bacterioides, Eubacteria and Peptostreptococcus are increased whereas Bifidobacteria numbers are reduced (Linskens et al., Scand J Gastroenterol Suppl. 2001; (234):29-40); in ulcerative colitis, the number of facultative anaerobes is increased. In these inflammatory bowel diseases, such microbial imbalances cause increased immune stimulation, and enhanced mucosal permeability (Sartor, Proc Natl Acad Sci USA. 2008 Oct. 28; 105(43):16413-4). In obese subjects, the relative proportion of Bacteroidetes has been shown to be decreased relative to lean people (Ley et al., Nature. 2006 Dec. 21; 444(7122):1022-3), and possible links of microbial imbalances with the development of diabetes have also been discussed (Cani et al., Pathol Biol (Paris). 2008 July; 56(5):305-9). In the skin, a role for the indigenous microbiota in health and disesase has been suggested in both infectious and noninfectious diseases and disorders, such as atopic dermatitis, eczema, rosacea, psoriasis, and acne (Holland et al. Br. J. Dermatol. 96:623-626; Thomsen et al. Arch. Dermatol. 116:1031-1034; Till et al. Br. J. Dermatol. 142:885-892; Paulino et al. J. Clin. Microbiol. 44:2933-2941). Furthermore, the resident microbiota may also become pathogenic in response to an impaired skin barrier (Roth and James Annu Rev Microbiol. 1988; 42:441-64). Bacterial vaginosis is caused by an imbalance of the naturally occurring vaginal microbiota. While the normal vaginal microbiota is dominated by Lactobacillus, in grade 2 (intermediate) bacterial vaginosis, Gardnerella and Mobiluncus spp. are also present, in addition to Lactobacilli. In grade 3 (bacterial vaginosis), Gardnerella and Mobiluncus spp. predominate, and Lactobacilli are few or absent (Hay et al., Br. Med. J., 308, 295-298, 1994).

Other conditions where a microbial link is suspected based on preliminary evidence include rheumatoid arthritis, multiple sclerosis, Parkinson's disease, Alzheimer's disease, and cystic fibrosis.

### Types of Organisms Present in Niches

The methods may be directed to relevant members of a bacterial network, including phyla relevant in the human microbiota, such as, but not limited to, the Bacteroidetes, and the Firmicutes, genus such as Bacteroides, Bifidobacterium, and Lactobacillus, and species, such as Bacteroides thetaiotaomicron or Faecalibacterim prausnitzii.

### X. Applications of Identified Interactions

The interactions identified by these methods may be used for diagnosis or prognosis of a condition, monitoring of a condition, and prevention, management, or treatment of a condition.

In one embodiment, a method for developing diagnostics for the determination of a physiological state or condition associated with the microbiota is provided, comprising (i) analyzing a biological interaction network within a superorganism which includes at least one microbial derived component, (ii) selecting a node, edge, or motif in the network based on one or more network parameters, and (iii) using a measure of the node, edge, or motif from a subject's sample (e.g. a urine, fecal, or blood sample) to either assess a subject's risk of developing a microbiota-associated disease, diagnose the presence of a microbiota-associated disease, select a course of treatment, or to assess the efficacy of a concomitant treatment. In another embodiment, the method comprises the additional step of (iv) validating the functional role of the node, edge, or motif by any of the perturbation methods (e.g. inhibition, knockout, supplementation, etc.) previously described.

In a further embodiment, the data set used to generate a biological interaction network for further analysis is generated via tandem affinity purification experiments or via yeast two hybrid screens. The size of the data sets generated typically exceeds a subject's ability to manually analyze the data sets, in which case analysis of the interaction network can be done automatically with an algorithm (See for example K Y Yip, H Yu, P M Kim, M Schultz, M Gerstein (2006) Bioinformatics 22: 2968-70) that returns only selected information, such as maximal motifs (a motif is maximal if adding a node to it without taking away any edges will render the motif no longer fulfilling the requirements)

In a further embodiment, the data used to generate the network is genetic or biochemical data of metabolic pathways in the microbes, used to create a microbiome community metabolic network. An example of such a genetic data-driven metabolic network can be found in Borenstein E and Feldman M W, 2009, J Comput Biol. February; 16(2):191-200. Such networks are used to probe relationships of a disease state to the perturbation of the networks. In one embodiment, the baseline “healthy” metabolic network is compared to a metabolic network representative of a “diseased” state, with the largest variations identified as as diagnostic markers of the disease and targets for therapeutic correction. In a further embodiment, the method of testing a therapeutic target comprises computationally perturbing highly-connected or centralized nodes or edges of the network, and observing shifts in the network, with shifts approaching the “normal” state identified as novel therapeutic strategies and targets.

An example of the use of a biological taxa-based network model for discrimination between disease and healthy states is described in Naqvi A et al, 2010, Chem. Biodivers. 7(5): 1040-50.

In a further embodiment, a method for developing microbiota modulators for the improvement of health is provided, comprising (i) analyzing a biological interaction network within a superorganism which includes at least one microbial derived component, (ii) selecting a node, edge, or motif in the network based on one or more network parameters, (iii) validating the functional role of the node, edge, or motif by any of the genetic knockout methods previously described, (iv) screening compounds in an in vitro or in vivo assay that models the interaction (for example, if the predicted interaction involves the consumption of a substrate by a bacterial enzyme, an in vitro fluorescence activity assay of the enzyme in the presence of the substrate may be used to validate the predicted interaction), and (v) selecting the most potent modulators of the node, edge, or motif.

In a further embodiment, identification of key interactions comprises comparing interactions from at least two separate data sets and selecting the interactions that experience the largest changes across the data sets. For example, samples from healthy and diseased individuals may be collected, followed by analysis and comparison of the samples and identification of the interactions that undergo the largest changes. The interactions then suggest a biomarker and/or a target for the disease. The largest changes can be individual points (nodes or edges) within the network, or more complex functions representing broader profiles of the network (e.g. the general network topology or connectivity pattern difference between healthy and diseased states can itself serve as a diagnostic or therapeutic target). Alternatively, two or more samples of interactions from one subject obtained at different points in time may be compared. The interactions undergoing measurable changes may reveal the presence of a developing microbiota-associated condition, or be used to track a subject's response to a treatment. In a preferred embodiment, the data sets include data selected from metagenomic, transcriptomic, or metabolic analysis. In a further embodiment, identification of a key interaction further comprises applying an external perturbation, wherein the perturbation may cause a change in the composition, absolute number of microbes, or metabolic activity of the microbiota. In a preferred embodiment, the perturbation is selected from (i) a change in diet, (ii) a pharmaceutical intervention (e.g. a microbe-directed agent such as an antibiotic, or a host-directed agent such as a human physiology-targeted drug), (iii) administration of a prebiotic nutritional supplement, (iv) administration of a probiotic, and (iv) administration of a synbiotic. Subsequently, measurements of the interactions before and after the perturbation are compared, and the nodes, edges, or motifs that experience the largest changes are selected.

Non-medical applications are also contemplated. In one embodiment, the microbial populations are in a soil and modulators are needed for applications such as waste remediation or alteration of crop yields. In another embodiment, the microbial populations are in a liquid phase (for example a pond or the medium of a bioreactor), and are used to produce a biofuel.

We claim:

1. A method for modulating microbiota comprising
(i) analyzing a biological interaction network within a superorganism which includes at least one microbial derived component, wherein a superorganism is an organism consisting of many organisms, and wherein a microbial derived component is a microbe, or a gene, protein, transcript, carbohydrate, lipid, or metabolite derived from a microbe,
(ii) selecting a node or edge in the network, wherein a node is a terminal point or an intersection point of a graphical representation of a network; and wherein an edge is a link between two nodes, and
(iii) providing modulators of the node or edge.

2. The method of claim 1, wherein the modulator is selected from the group consisting of a small molecule, a protein, a carbohydrate, a lipid, a phage, a prebiotic, a probiotic, and a commensal organism, and combinations thereof.

3. The method of claim 1, wherein analyzing comprises the step of building a model of a biological interaction network using a method selected from the group consisting of a probabilistic bayesian network model, linear algebraic equations, partial least squares, Principle component analysis, Boolean models, and Clustering models.

4. The method of claim 1, wherein the network is selected from the group consisting of a bacterial interaction network, a bacterial-host interaction network, a whole-organism level interaction network, a biochemical interaction network, and a signaling network.

5. The method of claim 1, wherein the node is selected from the group consisting of a bacterial cell, a bacterial species, a bacterial protein, a bacterial enzyme, and a bacterial metabolite, wherein a node is a terminal point or an intersection point of a graphical representation of a network.

6. The method of claim 1, wherein the node is selected from the group consisting of a host cell, a host protein, a host enzyme, and a host metabolite, wherein a node is a terminal point or an intersection point of a graphical representation of a network.

7. The method of claim 1, wherein the edge is selected from the group consisting of a catalytic transformation, a complex formation, a signal transfer, regulation by a protein-protein interaction, a protein phosphorylation event, regulation of an enzymatic activity, and production of a secondary messenger, wherein an edge is a link between two nodes.

8. The method of claim 1, wherein the node or edge is selected based on a network parameter indicating the highest ranked node or edge according to a measure of relative prevalence, connectivity, evolutionary similarity, density, centrality, clustering coefficient, structural equivalence, and path length, wherein relative prevalence is the number of occurrences of a node divided by the total number of occurrences of all other nodes in a network; wherein connectivity is a measure of the number of edge connections of a node to the rest of nodes in the network; wherein evolutionary similarity is a measure of the degree of shared ancestry between two or more nodes; wherein density is the proportion of connections in a network relative to the total number possible connections; wherein centrality is a measure of the relative importance of a node or edge in the network based on one of four measures comprising degree centrality (the number of links incident upon a node), betweenness (the number of times a given node appears in shortest paths between other nodes), closeness (the distance between two nodes) and eigenvector centrality (the principal eigenvector of the adjacency matrix of a network); wherein the clustering coefficient is a measure of the likelihood that two nodes connected to a given node are also connected themselves; wherein structural equivalence is a measure of the extent to which nodes have a common set of connections to other nodes in the system; and wherein path length measures the distances between pairs of nodes in the network, with shorter distances being assigned a higher ranking.

9. The method of claim 1, wherein the network parameter is a measure of covariance.

10. A method for developing diagnostics for the determination of a physiological state comprising
(i) analyzing a biological interaction network within a superorganism which includes at least one microbial derived component,
(ii) selecting a node or edge in the network based on one or more network parameters, and
(iii) developing a diagnostic to measure the node.

11. The method of claim 10, comprising obtaining a sample from the group consisting of aurin, fecal, plasma, blood, saliva, sputum, CSF, and biopsy based test sample, for analysis.
